(19) United States
(12) Patent Application Publication
(10) Pub. No.: US 2005/0070907 A1
(43) Pub. Date: Mar. 31, 2005

(54) Method and device for drilling and tapping a bore for a bone screw

(76) Inventor: Dennis L. Abernathie, Columbia, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/951,227

(22) Filed: Sep. 27, 2004

Related U.S. Application Data
(60) Provisional application No. 60/505,777, filed on Sep. 25, 2003.

(51) Int. Cl. A61B 17/32

(52) U.S. Cl. 606/80

(57) ABSTRACT

The present invention is directed to a device for drilling and tapping a bore for receiving a bone screw. The device includes a drill tip portion for creating a bore, a threaded shank portion for creating female threads within the bore so that the bore can receive a bone screw, and a handle or attachment portion for the purpose of manipulating the device. The present invention is also directed to a method of using the device.

### CROSS REFERENCE TO RELATED APPLICATIONS

This Application claims benefit of U.S. Provisional Application No. 60/505,777, filed Sep. 25, 2003.

### BACKGROUND OF THE INVENTION

Bone screws are often used for stabilizing bones, internal fixation of fractures, and attaching orthopaedic implants. Bones screws are generally made of metallic material, such as titanium, cobalt-chrome alloys, and stainless steel. The orthopaedic procedure for starting, drilling, and tapping a hole for insertion of a bone screw typically involves a number of steps. Once the site for the bone screw is visualized, a surgeon will expose the fracture or osteotomy site. With the advent of x-rays and other visualization techniques, the exposure area required to place a bone screw may be reduced and thus the procedure can be done percutaneously. When percutaneous or other exposures are used, the procedure often begins with the placement of a guide wire at the intended screw bore location to provide a reference point for application of tools used for starting, drilling, and tapping the screw hole. If a guide wire is not utilized to maintain the reference point between the application of these separate tools, as the tools are exchanging, the bore location or alignment is often lost. After the firm guide wire is placed at the intended screw location, its position is confirmed. Then, a tube is placed over the wire to start the screw bore. Guided by the tube, the surgeon will then use a drill to drill a bore into the bone to accommodate the bone screw. Next, another device is used to tap the bore to provide a female thread therein. Finally, yet another device is used to insert the bone screw by rotating the bone screw to threadably engage the female threads to attach the bone screw to the bone. After the fracture has sufficiently healed, it is sometimes desirable to remove the bone screw.

Although acceptable results have been achieved with the above-noted process, it would be desirable to modify the above-identified procedure to reduce instrumentation, decrease the number of required steps, increase accuracy of the procedure, and reduce costs. Additional objects of the present invention are set forth below.

### SUMMARY OF THE INVENTION

The present invention relates to a novel drill-tap device and method for localizing, starting, drilling, and tapping the intended site of bone screw insertion in a single operation, using a single tool. By radiographic visualization, or other technique, the distal end of the drill-tap device is placed in the proper starting location on the surface of the bone, either by penetration of the skin for cutaneous procedures or by dissection to the surface of the bone for open procedures. The shaft of the drill-tap device is aligned in proper orientation. A tool, such as a hammer, is used to impact the proximal end of the drill-tap device, thereby driving the distal cutting tip of the drill-tap into the bone and creating a small bore in the bone at the desired location. The drill-tap device can be rotated by hand or with an instrument, such as a drill. By rotating the drill-tap device, the bore is enlarged and sized to the root diameter of the intended bone screw. As the drill-tap device progresses into the bone, the device cuts threads into the surface of the bore to facilitate bone screw insertion. The drill-tap is then removed and a bone screw is placed in the bore formed by the drill-tap device. Speed and reduced instrumentation are substantial advantages of this device. Also, by drilling and enlarging the bore with a single device, correction of alignment may be achieved before the final bore is made.

Further, the device tends to direct itself toward the intended hole orientation. In the placement of pedicle screws, the pedicle is targeted. After penetration, the slope of the advancing drill tip enlarges the bore and the sides of the drill tip begin to contact the inner wall of the pedicle. Since the density is greater at the cortical bone of the wall, the device tends to penetrate the softer cancellous bone of the pedicle. Similarly, when installing stabilization screws into intermedullary devices, such as a femoral rod, the smaller tip of the drill-tap device finds the hole of the rod after cortical penetration. As the drill-tip device progresses further, the path of least resistance is through the hole of the rod and the enlarging drill tip point centers itself within this hole.

Furthermore, image guiding technology that aids in the placement of bone screws with minimal use of x-rays can also be utilized. The drill-tap device can either be used independently or with the assistance of image guiding technology, such as ultrasound, video, electromagentic waves, or infrared light.

The present invention can also be used to increase the effectiveness of a surgical retractor. A bone screw hole is drilled into the bone at a point where a retractor would be of optimum benefit. The head of the screw, formed to accept the retractor, is inserted into the hole. By anchoring the retractor against the fixed point of screw placement, the retractor is less likely to slip than on an irregular bony surface. After the surgical procedure, the screw can be removed.

Additionally, if desired, a screw of smaller diameter can be placed into a pedicle for visualization during spinal surgery. When it is no longer needed, the screw can be removed and, after proper preparation, this bore can be filled with a pedicle screw.

### BRIEF DESCRIPTION OF THE DRAWINGS

FIG. 1 illustrates various aspects of one embodiment of the present invention. FIG. 1(a) is a close view of the tip of one embodiment of the present invention as seen with the point of the tip aligned directly toward the viewer; FIG. 1(b) provides a side view of one embodiment of the device of the present invention.

### DETAILED DESCRIPTION OF THE INVENTION

Referring now to the drawings, wherein like numeral indicate like parts, the numeral 10 refers generally to a device constructed in accordance with the teachings of the present invention. Drill-tap device 10 has a generally pyramid-shaped drill tip 14, formed of three cutting edges or flanges 15. Although a pyramid-shape with three flanges is shown in the drawings, this arrangement is exemplary of one embodiment of the present device. It is contemplated that other arrangements, such as a traditional bayonet-point or other geometry, including multiple flanges, could also be used.

A point 12 at the distal end of device 10 is designed to penetrate bone and initiate a bore therein. After the shaft 16 of drill-tap device 10 is aligned in the proper direction, a force from a tool such as a hammer is applied against the proximal end of drill-tap device 10, thereby driving distal point 12 into the bone and creating a small bore in the bone at the desired location. By rotating drill-tap device 10, manually or with a device such as a drill, the bore is enlarged and sized to the root diameter of the intended bone screw. A reamer 18 forms a more circular bore and allows drill-tap device 10 to penetrate the bore deeper without further enlargement of the bore. During this process, realignment of drill-tap 10 is performed until the desired direction is achieved. Realignment is preferably performed while rest portion 19 of device 10 is situated within the bore. Rest portion 19 is smooth and will not disturb the bore during the realignment step. If a completely circular bore is not necessary, reamer portion 18 of drill-tap device 10 can be omitted.

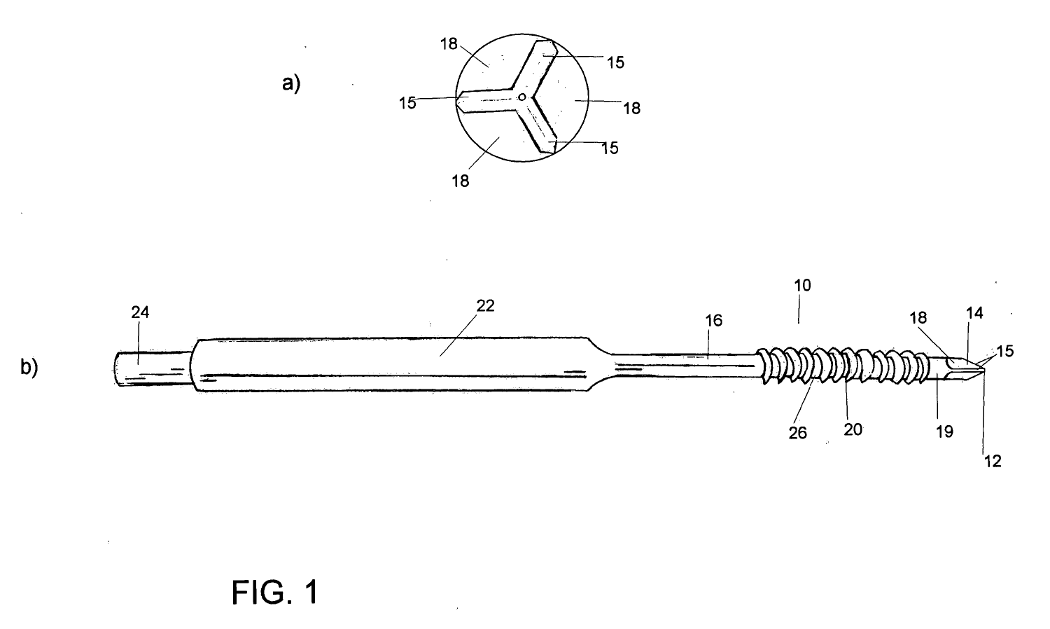

The tap portion 20 of device 10 is introduced after reamer 18 (if reamer 18 is not necessary, however, use of tap portion 20 can commence immediately after tip 14). Tap 20 is essentially a threaded shank portion fashioned to correspond to the thread configuration of the bone screw to be utilized. In FIG. 1, for example, tap portion 20 includes the area in which screw threads are illustrated. The screw thread in FIG. 1 is illustrative only and is not meant to correspond to any specific bone screw. The precise pattern, arrangement, size and spacing of such threads will vary depending on which bone screw is to be used in the bore created by the present device. A drill-tap device 10 is selected from a plurality of drill-tap devices with differing lengths, widths, and threads, depending on the size of the screw, the diameter of the screw, the length of the screw, the number of threads per inch, and the width of the thread, among other things.

When drill-tap device 10 is rotated, tap 20 is rotated into the bore to provide a female thread therein. Tap portion 20 of drill-tap device 10 preferably incorporates one or more grooves 26 that run the entire length of tap 20 and create discontinuous threads in tap portion 20 of device 10. Grooves 26 permit the carved-out bone to be removed from the bore. FIG. 1 illustrates a drill-tap device 10 with grooves 26 having a curved profile, but other groove profile geometries, including grooves having profiles of right triangles, or a combination of varying geometries, can also be used.

After tap 20 comes shaft 16, which incorporates either an attachment for a guiding instrument or a handle, or other manipulation device that facilitates for the rotation of drill-tap device 10 and advancement of device 10 into its intended target. Shaft 16 may widen to form a gripping portion 22, and end in an attachment portion 24, may include only a gripping portion 22 or only an attachment portion 24, or may include neither of these. Drill-tap device 10 can be rotated manually or with a drill or similar driving tool. After the desired depth is reached, drill-tap device 10 is removed and a bone screw is placed in the bore formed by drill-tap device 10 by rotating the bone screw to threadably attach the bone screw to the bone.

A T-handle may be used as a manipulation device. Such a handle generally has a flat area located above the t-shaped portion of the handle for the purpose of receiving a force or impact from a tool, such as a mallet. Impact against the flat area allows a hole to be started without loss of alignment, and further protects the t-handle from impact. The t-handle is then rotated in order to advance drill-tap device 10 into the bore. Any other suitable manipulation device known in the art could also be used. The t-handle or other manipulation device preferably engages drill-tap 10 at attachment portion 24.

Drill-tap device 10 may also be used to conduct separate operations of penetrating, drilling, and directing for other purposes, such as placing a cannula.

In another embodiment, a drill-tap device constructed in accordance with the teachings of the present invention can be configured as a guide and alignment tube manufactured in combination. The cutting tip is formed onto the guide and continues onto the alignment tube. The combination device is penetrated, drilled, and advanced into bone. The inner guide is removed and the outer tube remains. Such a device could be advanced for biopsy, or could be used to inject cement for vertebroplasty.

In yet another embodiment, a drill-tap device may be detachable removed from the guiding instrument or handle and can remain in the desired location to stabilize a bone, reattach a fragment, or attach an orthopaedic device. In this embodiment, tip 14 and tap portion 20 function as a bone screw.

In yet another embodiment, a drill-tap device may be fashioned as part of an implanting device, such as a self-tapping screw. The screw penetrates the bone and is aligned appropriately. As it advances, the screw drills its own hole and taps its own thread for fixation. If a thread configuration is undesired, other methods of fixation, such as riveting, could be utilized.

By improving and simplifying the procedure for forming a bore for a bone screw, a drill-tap device constructed in accordance with the teachings of the present invention also improves the procedure for utilizing a bone screw to stabilize a surgical retractor. A temporary screw is penetrated, drilled, aligned, and advanced into bone near the area that will be retracted. The head of the screw is formed to accept and hold the retractor. Such a formation is more stable than levering against bone. After the procedure, the temporary bone screw is removed.

While embodiments of the invention have been described above and demonstrated by the drawing, variations of the present invention will be apparent to those skilled in the art upon reading this disclosure. The invention should not be construed as limited to the specific forms described and shown herein, but should be limited only by the claims that follow.

1. A device for drilling and tapping a bore comprising:
a) a drill tip portion having at least one cutting edge;
b) a threaded shank portion having distal and proximal portions at opposite ends thereof, extending longitudinally from said drill tip portion beginning at the proximal portion of said threaded shank portion; and
c) a shaft portion extending longitudinally from said threaded shank portion.

2. A device according to claim 1 wherein said at least one cutting edge of said drill tip portion is a flange.

3. A device according to claim 2 wherein said drill tip portion is generally pyramid shaped and comprises at least three flanges.

4. A device according to claim 1 further comprising a reamer portion extending distally from said drill tip portion and between said drill tip portion and said threaded shank portion, such that a smooth circular bore is provided when said device is utilized in drilling a bore.

5. A device according to claim 1 further comprising a handle portion extending longitudinally from said shaft portion.

6. A device according to claim 5 further comprising an attachment portion extending longitudinally from said handle portion.

7. A device according to claim 1 further comprising an attachment portion extending longitudinally from said shaft portion.

8. A device according to claim 1 wherein said threaded shank portion is adapted to provide a threaded bore for receiving a bone screw.

9. A device according to claim 1 wherein said threaded shank portion is adapted such that the threads of said threaded shank portion are discontinuous.

10. A device according to claim 1 further comprising a rest portion for use in realignment of said device.

11. A method of drilling and tapping a bore in bone comprising:
a) contacting said bone with a tip of a drill tip portion of a device that includes a drill tip portion and a threaded shank portion;
b) rotating said device such that said drill tip portion creates a bore in said bone;
c) introducing said threaded shank portion of said device into said bone and further rotating said device such that said threaded shank portion creates female threads within said bore.

12. The method of claim 11 further comprising the step of realigning said device between steps b) and c) enumerated above.
